(19) United States
(12) Patent Application Publication
(10) Pub. No.: US 2026/0137457 A1
(43) Pub. Date: May 21, 2026

(54) SYSTEMS AND METHODS FOR SURGICAL INSTRUMENTS NAVIGATION USING PERSONALIZED DYNAMIC MARKERS

(71) Applicant: Shmuel BEN MUVHAR KAHANA, Peduel (IL)

(72) Inventor: Shmuel BEN MUVHAR KAHANA, Peduel (IL)

(21) Appl. No.: 19/121,812

(22) PCT Filed: Oct. 16, 2023
(86) PCT No.: PCT/IL2023/051078

§ 371 Date: Apr. 17, 2025

Related U.S. Application Data
(60) Provisional application No. 63/380,236, filed on Oct. 20, 2022.

(51) Int. Cl. A61B 34/20; A61B 34/00; A61B 34/10; A61B 90/00

(52) U.S. Cl. CPC A61B 34/20; A61B 34/25; A61B 90/37; A61B 2034/101; A61B 2090/374

(57) ABSTRACT

Systems and method for navigation of surgical instruments through an inner body part by enabling to: obtain biometrical properties of a hand of a specific surgeon; ongoing measure updated properties of the hand of the specific surgeon during the surgical procedure to determine an updated hand position of the hand of the surgeon within a coordinate system, based on the measured updated properties and the obtained biometrical properties of the specific surgeon; and determine updated relative position between the surgical instrument being used and the body part, based on an updated image of the body part and determined updated hand position of the specific surgeon within the coordinate system, for enabling using the hand of the specific surgeon as a personalized dynamic marker for assisting the surgeon in navigation of the surgical instrument held thereby, during the surgical procedure of the body part.

### TECHNICAL FIELD

The present invention generally relates to systems and methods for assisting surgeons in navigation of a surgical instrument during a surgical procedure and more particularly to systems and methods for navigation of a surgical instrument using dynamic markers.

### BACKGROUND

Medical imaging is a wide term relating to any medical diagnostic, therapeutical, surgical and/or prognostic system or process that uses one or more imaging systems or devices that produce 2D or 3D imagery information of internal and/or external body parts, such as magnetic resonance imaging (MRI) systems, computerized tomography (CT) systems, ultrasound-based systems, X-ray systems/devices, isotopic tomography systems, endoscopic systems, etc.

Imagery systems are typically configured to produce one or more images indicative of anatomical properties of organs, body parts, body systems and/or physiological characteristics thereof.

When conducting a surgical procedure, involving intrusively penetrating of one or more surgical tools to a body of a patient, e.g., for operating an internal organ/tissue of the patient and/or for performing an intrusive diagnosis/detection/imaging (e.g., using endoscopic systems), the positioning (i.e., location within a 3D coordinate system and orientation) of the edge of the surgical tool being used to perform the operation should be constantly and controllably moved by the surgeon performing the operation, in order to reach a different zone/area/spot/circumference of the inner body part being operated at each given moment and to avoid/reduce erroneous adversities by having the edge of the surgical instrument engaged with inner tissue that is not to be operated, irradiated and/or detected. In many cases, any slight error in the instrument's positioning may lead to sever and often irreversible outcomes.

To reduce such impairments, the internal area/organ to be operated/tested is often imaged in real time during the performance of the surgical procedure, using one or more fixed markers attached or marked over the patient's body or placed externally to the patient. These fixed markers are also detectable by the imaging system to identify the positioning of the operated internal organ/tissue/area for identifying position of the operated organ/area/tissue in respect to a fixed coordinate system. The one or more fixed markers allow identifying the position of the patient's external body part or body at each given moment in respect to the fixed coordinate system and thereby deduce or estimate the position of the operated organ/tissue/area in respect to the surgical tool position. This technique can provide sufficient accuracy when dealing with operated body tissues, organs and/or areas that are likely to always be positioned in the same exact manner relative to the positioning of the larger external body part of the patient and in cases in which the surgical tools being used are not very sensitive to erroneous positioning thereof.

However, such techniques do not provide a sufficient solution when operating on elastic tissues such as soft tissue and/or when operating on tissues of varying elasticity levels as well as for operations using surgical tools and/or procedures that are highly sensitive to errors in the positioning of the surgical tool or part thereof. Moreover, the above-mentioned marking techniques may also cause inaccurate detection of the position of the surgical tool and/or part thereof, in respect to the actual position of the specific area in the body part being operated, which may lead to catastrophic outcomes such as removing of a wrong section of the tissue or part thereof. For example, in cases of removal of malignant, pre-malignant or benign tumor tissues, requiring the removed tissue volume or area to be larger than the area/volume of the tumor, to reduce chances of tumor cells from being cut off and released into other tissues or into the patient's blood stream, errors in the positioning of the surgical tool or edge thereof may lead to leaving some of the tumor's tissue unremoved or cut through, which may lead to fatal outcomes.

In addition to the solutions presented above, there are solutions that involve the use of marker-marked surgical tools such as visual markers and/or electromagnetic markers, which enable monitoring the surgical tools being used, by tracking the markers associated with them. However, in these solutions, the markers of the marker-marked surgical tools must be in direct contact with or visible to designated detection means such as cameras, other optical sensors, electromagnetism detectors and the like.

### SUMMARY

Aspects of disclosed embodiments pertain to a method for assisting surgeons in surgical navigation of surgical instruments through an inner body part, the method may include, for example, at least:
(i) obtaining one or more biometrical properties of a hand of a specific surgeon, measured by using one or more sensors; and
   at each given moment or time frame of a surgical procedure conducted by the specific surgeon:
(ii) receiving at least one updated image of a body part to be operated, at a specific perspective of the body part within a predefined coordinate system;
   measuring one or more updated properties of the hand of the specific surgeon during the surgical procedure and determine an updated hand position of at least part of the hand of the specific surgeon within the coordinate system, based on the measured updated properties and the obtained biometrical properties of the hand of the specific surgeon; and determining updated relative position between the surgeon's hand and the body part, based on received updated image of the body part and determined updated hand position of the specific surgeon within the coordinate system, thereby using the hand of the specific surgeon as a personalized dynamic marker for assisting the surgeon in navigation of at least one surgical instrument held by the hand of the specific surgeon, during the surgical procedure of the body part.

Aspects of disclosed embodiments pertain to a system for assisting surgeons in surgical navigation of surgical instruments through an inner body part, the system comprising at least:
(i) one or more sensors at least for obtaining of one or more biometrical properties of a hand of a specific surgeon;
(ii) at least one processor configured at least for:
receiving an updated image of a body part at a specific perspective of the body part within a predefined coordinate system;
detecting one or more updated properties of the hand of the specific surgeon during the surgical procedure and determine an updated hand position of at least part of the hand of the specific surgeon, based on the detected updated properties and the obtained biometrical properties of the hand of the specific surgeon; and
determining updated relative position between the surgeon's hand and the body part, based on received updated image of the body and determined updated hand position of the specific surgeon, thereby using the hand of the specific surgeon as a personalized dynamic marker for assisting in guiding the specific surgeon through surgical procedure of the body part.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIGS. 1A-1F show schematic illustrations of a surgeon's hand in several postures and perspectives holding or not holding different surgical tools for obtainment various personal biometrical properties associated with the specific hand of the specific surgeon: FIG. 1A shows a first perspective view showing the back of a hand of a surgeon; FIG. 1B shows a second perspective view showing a front inner view of the hand of the surgeon; FIG. 1C shows a third perspective view showing the hand of the surgeon when holding a scalpel surgical instrument; FIG. 1D shows a fourth perspective view showing the hand of the surgeon when holding a different surgical instrument; FIG. 1E shows an open hand side view of the hand of the surgeon; and FIG. 1F shows the hand in the open hand view holding a surgical instrument such as a drilling tool having a handle and an elongated drilling arm;

FIG. 2 is a block diagram, schematically illustrating a system for assisting surgeons in surgical navigation of surgical instruments during surgical procedures by using hand(s) of a surgeon as a personalized dynamic marker, according to some embodiments;

FIG. 3 shows one exemplary interface of the system of FIG. 3, according to some embodiments, for enabling real time or near real time display of a combined 3D imagery of the operated body part or an area thereof and at least part of the surgical instrument proximal to the operated body part, based on estimated relative positioning therebetween, estimated by using the hand of the surgeon (holding the respective surgical instrument) as a personalized dynamic marker.

FIG. 4 is a flowchart schematically illustrating a method for assisting surgeons in surgical navigation of surgical instruments during surgical procedures by using hand(s) of a surgeon as a personalized dynamic marker, according to some embodiments.

The drawings in the above listed figures only provide schematic illustrations of elements, operations, functionalities, devices, etc. and do not necessarily encompass all operational steps, elements, devices, etc. required in order to enable the system, method or part thereof to perform its full functioning or purpose, but merely provide a scheme according to which a person with skills in the art can understand how to implement at least some main principles of the system/method.

Elements or object shown in the drawings of the figures listed above are not necessarily drawn to scale.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “obtaining”, “identifying”, “performing”, “providing”, “receiving”, “generating”, “processing”, “moving”, “estimating” deducing”, “associating”, “analyzing”, “processing” “repeating”, “determining”, “instructing”, “displaying”, “storing”, “saving”, “modifying”, “identifying”, “detecting”, “learning”, “adjusting”, “changing”, “simulating” and the like and/or any conjugations of such terms, include action and/or processes of a computer/controller/processor that manipulate data and/or transform data into other data, said data represented as physical quantities, e.g., such as electronic quantities, and/or said data representing the physical objects.

The terms “computer”, “processor”, “processing resource”, “processing circuitry”, and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

Aspects of disclosed embodiments pertain to systems and methods for assisting surgeons in navigation of surgical instruments through an inner body part by using personal biometrical properties of one or more hands of the specific surgeon in order to determine updated positioning (location and orientation) of the surgical instrument being used by the specific surgeon during the specific surgical procedure, thereby using the personal biometrical properties of the surgeon's hand(s) as a dynamic personal marker.

The term “hand” used herein may refer to any part of a hand limb that includes at least the hand palm.

Using the surgeon's hand(s) as a dynamic marker may be especially (yet not exclusively) advantageous when in cases in which the surgeon's hand (palm) is mostly external to the body of the patient during the surgical procedure, while the edge (tip) of the surgical instrument being used is internally located (inside the patient's body) and in cases in which maximal precision is required in the position of the edge (tip) of the surgical instrument especially when this edge is not visible to the surgeon's eye. Foe example in cases such as a dental procedure in which the drill tip is not visible to the dentists while precision of the relative drilling location precision in respect to the tooth/gum area to be drilled, at any given moment, is of high importance. In these cases, the positioning of the edge of the surgical instrument cannot be measured by simple detection means such as a camera located externally to the patient, making it very difficult to accurately detect the exact positioning of the edge of the surgical instrument in a predefined set 3D coordinate system and its relative positioning in respect to the tissue being operated. In these cases, external detection of the hand of the specific surgeon and comparing its positioning, posture and other properties, to previously acquired biometrical properties of the surgeon's hand and other information (measured in real time or previously acquired) indicative of the location and positioning of the body part being operated, will allow determining of the positioning of the edge of the surgical instrument held by that hand of the specific surgeon in the 3D coordinate system and in relation to the positioning of the tissue being operated.

The solution of using the specific hand of the specific surgeon as a personalized dynamic marker during surgical procedures, may be more accurate and advantageous than setting a fixed marker over a handle or any other external part of the surgical instrument being used since each surgeon applies different pressures and other force vectors over the same instrument resulting in different influences over the operated tissue (especially for soft tissue), yet such differences may not be measurable by simply tracking a fixed marker over the surgical instrument. The personalized hand dynamic marker may also enable using the system as a tool for training surgeons and/or testing surgeons' surgery skills.

Any one of the terms: “surgical instrument” or “surgical tool”, which may be interchangeably used herein, may refer to any device, object, equipment, etc., that has a part thereof or connected thereto that is controllably holdable by hand and that can be used for any purpose during a surgical procedure. For example, the surgical instruments may be used for cutting internal and or external tissue, illumination/radiation of internal and or external tissue (e.g., for using a laser beam to cut through body tissue or to illuminate an internal body part area/tissue for detection, navigation and/or diagnostical purposes), drilling, stitching, and/or connecting of tissues, etc.

The terms “surgical instrument(s)”, “instrument” and “surgical tool” may be used interchangeably herein and may refer to any element, device, apparatus, object and/or system that is used for any medical purpose such as for irradiating or illuminating, cutting, impinging, stimulating, detecting, stitching, marking, contracting/expanding, implanting an object, removal of tissue, etc. of/to/from the body part or part thereof.

The term “surgical procedure” or “operation” may be used interchangeably herein and may refer to any intrusive medical procedure for any medical purpose such as for any therapeutical and/or diagnostical purposes.

The term “surgical procedure” may also refer to virtual or simulated intrusive medical procedures done as a simulation over a real or virtual model of a body part.

The term “hand” used herein may refer to any part of a human hand that includes its autopodium part i.e., the hand palm or any one or more part thereof.

According to some embodiments, the term “body part” used herein may refer to any part of a body of an individual (human or animal) such as an internal organ, tissue, limb, bone, vessel, system or part thereof (such as digestion system, nervous system, etc.), etc., that can be subjected to any type and method of a surgical procedure for any purpose such as for any therapeutic and/or diagnostic purpose, and may also refer to a virtual or simulated body part used for surgeon's training and/or performances-evaluation.

The term “surgeon” used herein may refer to any person who performs any part of a surgical procedure whether a medical staff member (a certified surgeon, a nurse, an intern, a technician etc.) and is not limited only to certified surgery physicians.

According to some embodiments, there is provided a method for surgical instruments'navigation assistance, where the method may include at least the following steps:
(i) obtaining one or more biometrical properties of a hand of a specific surgeon, measured by using one or more sensors; and
at each given moment or time frame of a surgical procedure conducted by the specific surgeon:
(ii) receiving at least one updated image of a body part to be operated, at a specific perspective of the body part within a predefined coordinate system;
(iii) measuring one or more updated properties of the hand of the specific surgeon during the surgical procedure and determine an updated hand position of at least part of the hand of the specific surgeon within the coordinate system, based on the measured updated properties and the obtained biometrical properties of the hand of the specific surgeon; and
(iv) determining updated relative position between the surgeon's hand and the body part, based on received updated image of the body part and determined updated hand position of the specific surgeon within the coordinate system, thereby using the hand of the specific surgeon as a personalized dynamic marker for assisting the surgeon in navigation of at least one surgical instrument held by the hand of the specific surgeon, during the surgical procedure of the body part.

According to some embodiments, there is provided a system for assisting in navigation of surgical instruments during surgical procedures, where the system may include at least:
(i) one or more sensors such as optical sensors, at least for obtaining of one or more biometrical properties of at least one hand of a specific surgeon;
(ii) at least one processor configured at least for:
receiving an updated image of a body part at a specific perspective of the body part within a predefined coordinate system;
detecting one or more updated properties of the hand of the specific surgeon during the surgical procedure and determine an updated hand position of at least part of the hand of the specific surgeon, based on the detected updated properties and the obtained biometrical properties of the hand of the specific surgeon; and
determining updated relative position between the surgeon's hand and the body part, based on received updated image of the body and determined updated hand position of the specific surgeon, thereby using the hand of the specific surgeon as a personalized dynamic marker for assisting in guiding the specific surgeon through surgical procedure of the body part.

According to some embodiments, the biometrical properties of the hand of the specific surgeon may be any physical and measurable personal characteristics of the specific hand of the specific surgeon such as, for example one or more of:
topography of the hand of the specific surgeon; one or more topographies of typical hand postures of the outer surface of the specific hand of the specific surgeon or part thereof, e.g., when holding one or more types and sizes of surgical instruments, where the hand topography may be represented and stored as one or more 2D (two-dimensional) or 3D images/models;
2D and/or 3D images/models of typical movements of the hand of the specific surgeon when holding and using one or more types and sizes of surgical instruments;
specific anatomical properties of parts of the hand such as specific length, shape, size, topography and/or width of one or more different parts of the hand of the specific surgeon, such as length, shape and width of each finger part of each finger (finger part being defined as covering a different finger-phalanx) when the hand is at a relaxed state applying no pressure and optionally also at different states when applying different pressure over an object such as a handle of a surgical instrument (causing for instance the fingers pressing the instrument handle to contract in width, etc.);
relative position and/or distance between one or more parts of the hand of the specific surgeon;
relations between the posture of the hand of the specific surgeon and a relative directionality of the surgical instrument or a edge thereof (herein “pointing direction” or “pointing vector direction”) held by the hand of the specific surgeon or part thereof;
changes in one or more of the one or more biometrical properties of the hand of the specific surgeon when under various conditions such as various postures, positions, applying of different pressures over different instruments, etc.

According to some embodiments a 3D model of at least part of the hand of the specific surgeon may be generated, where the 3D hand model of the specific surgeon may be based on the obtained one or more biometrical hand properties of the specific surgeon.

The 3D hand model of the specific surgeon may then be used during a surgical procedure (operation) by comparing this 3D model with real time detected imagery of the hand of the specific surgeon, in order to determine updated hand properties such as updated relative position between the hand of the specific surgeon and the body part being operated by him/her, physical parameters values such as torque and/or pressure applied by the surgeon over the instrument, ultimately for determining the corresponding updated position of at least part of a surgical instrument held by the specific surgeon in respect to at least an area of the body part (such the exact location and orientation of a edge portion of the surgical instrument in respect to a body part positioning, in order to have the instrument operate the body part tissue area required to be operated (e.g., cut, illuminated, irradiated, stitched, inserted with a stent or a catheter etc.).

According to some embodiments, the system may include one or more software and/or hardware based machine-learning modules, algorithms, programs and/or tools for measuring of the relation(s) between measured behavior of the biometrical (physical) properties of the hand of each specific surgeon, the typical behaviors of the hand during surgery and when using different surgical instruments, and surgeon's hand related behaviors implications/influences over the physical properties of the surgical instrument or part (e.g., edge) thereof and over the tissue (body part) being operated, for each surgical procedure type and each stage within that particular procedure, and for each type of body part and tissue properties (such as tissue type, elasticity/hardness level etc., estimated or simulated organ/tissue/tumor periphery, etc.).

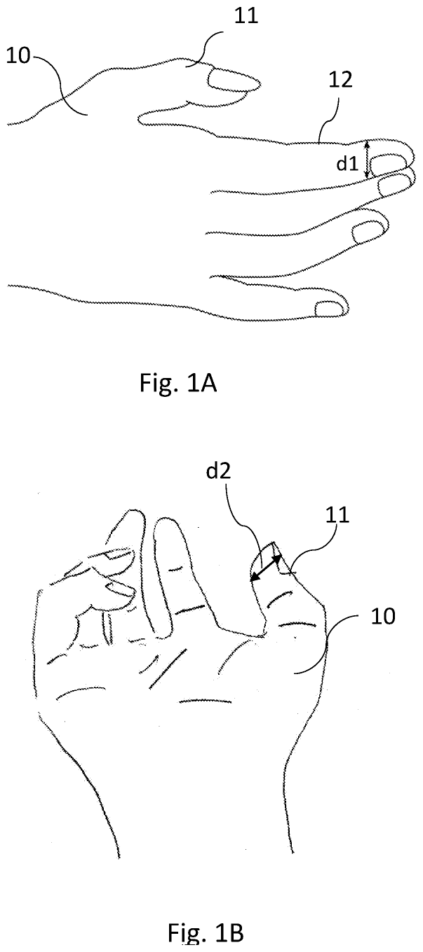

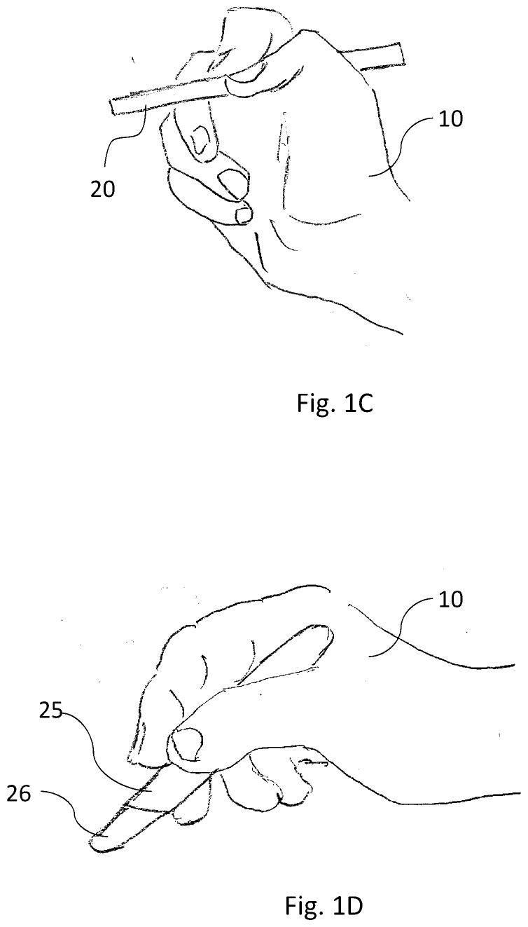

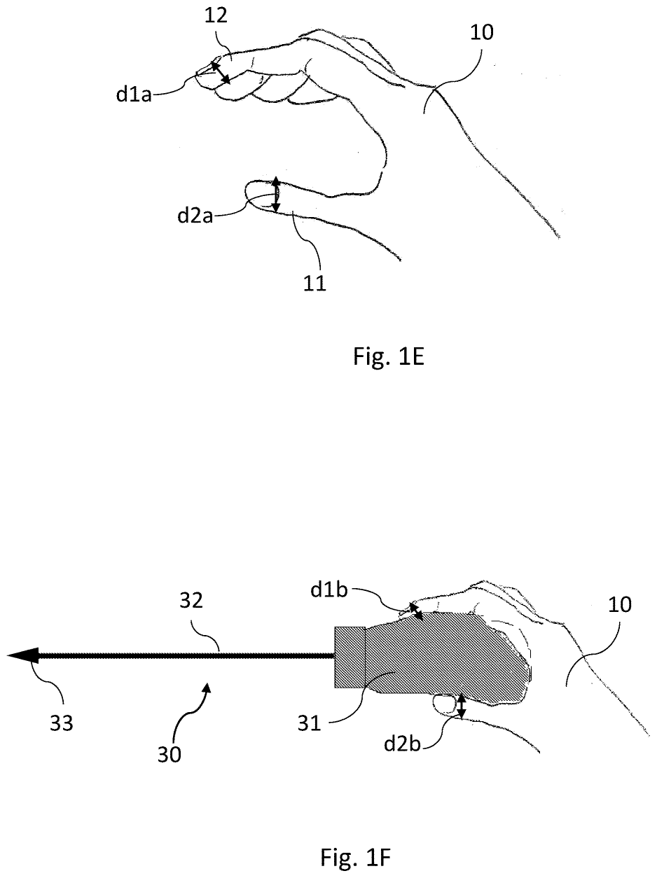

Reference is now made to FIGS. 1A-1F showing a schematic illustration of a right hand 10 of a specific surgeon in various postures and perspectives and when not holding or holding different surgical instruments.

According to some embodiments, several biometrical properties may be obtainable by taking multiple still 2D and/or 3D images of the surgeon's hand 10 such as the overall shape and size of the hand 10 (e.g., by using one or more sensors such as one or more cameras, a 3D point cloud laser detection device, a laser scanner device, etc.), specific shapes, lengths, widths and/or overall sizes of one or more of the hand's fingers such as the thumb 11 and the index finger 12 and/or relative length/size therebetween such as the ratio between the width d1 of the pad of the index finger 12 and the width d2 of the pad of the thumb 11 (see FIGS. 1A-1B) when the hand is in a loose state (where none of the fingers is contracted or squeezed to any other object or hand part (also shown in FIG. 1E)). Additional or alternative images/models/postures/states of the surgeon's hand may be obtainable such as one or more states of the hand 10 when holding different surgical instruments such as tools 20 (FIG. 1C), 25 (FIG. 1D).

As shown in FIGS. 1E and 1F, thickness d1a of the index finger 12 and thickness d2a of the thumb 11 of the right hand 10 of the specific surgeon in a specific posture (taken from one or more perspectives) that is typical for holding a handle 31 of a specific surgical instrument 30, are measured without the instrument 30 being held. One or more images of the right hand 10b of the specific surgeon is acquired also when holding the instrument 30 (which may include the handle 31, an elongated and optionally elastic middle section 32 and an edge functional tip 33 that is designed to engage with the body part tissue) and optionally when applying various pressures over the handle 31 of the surgical instrument 30, in various perspectives, in various instrument orientating angles, etc., for determining for each posture, thicknesses d1b and d2b of the index finger and thumb, respectively. This may enable determining associations between the thickness of one or more of the fingers 11 and 12 in relation to one or more characteristics of the specific surgical instrument 30b being used such as the instrument's 30 positioning, the 3D point/area location of the instrument's tip 33 and 3D direction of its pointing vector, the functionality quality and/or state of the instrument 30 etc. These determined characteristics, deduced from determined changes in thickness of one or more fingers of the surgeon, may enable estimating in real time during surgery, various characteristics of the surgery itself, the surgeon's performances and/or the functionality state and performances level of the instruments being used during the surgical procedure, such as for example, one or more of the following features/parameters/properties and/or analysis of such:
relative position of the edge of the instrument being used in respect to the body part tissue;
surgery state, conditions and/or performances quality;
instrument's state (3D position, directionality, mode of operation, etc.), conditions, and/or performances/functionality quality and/or state;
applied pressure and/or applied action over the tissue at each given moment;
identification of hazardous situations and outputting of indication thereof.

A 3D model of the surgeon's (right) hand 10 may be generated for at least one of the postures measured via the one or more sensors to be later used for detecting updated surgeon's hand posture, positioning, properties at each given moment or timeframe during the surgical procedure.

According to some embodiments, this preliminary process of acquiring/obtaining of one or more of the biometrical properties of each hand of each surgeon may result in a storage of “a surgeon's data” indicative of the measured biometrical properties, this surgeon's data may be associated (e.g., by way of database data-structure) with the specific hand of the specific surgeon and may comprise one or more of: at least one 3D model of the surgeon's hand and/or part thereof and/or the hand when holding one or more instruments, values of biomaterial measurable parameters such as lengths/widths of fingers, lengths/widths of finger sections, hand parts proportions, etc.

In some embodiments, the shape and dimensions of the finger nail of one or more of the surgeon's fingers may be used at least as one of the dynamic markers of the hand 10 as the orientation and location of the nail in the 3D coordinates system may be easy and quick to identify based on e.g., video camera detection operable during the surgery procedure.

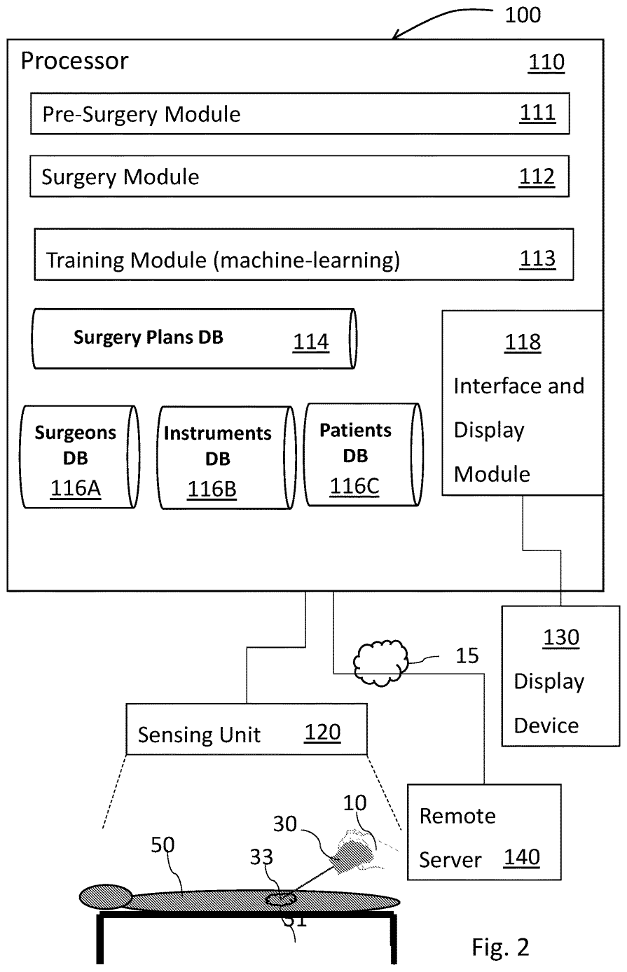

Reference is now made to FIG. 2 showing a block diagram of a system 100 for assisting navigation of a surgical instrument, according to some embodiments. The system 100 may include for example: a processor 110, at least one sensing unit 120, a display device 130, and optionally also one or more remote processing units such as a remote server 140.

According to some embodiments, the sensor unit 120 may include one or more optical sensors such as one or more stills and/or video cameras, configured and locatable to be used for the preliminary measuring of the one or more biometrical properties of one or more hands of the surgeon and for ongoing monitoring and imaging of the surgical procedure.

According to some embodiments, the processor 110 may be configured to receive store, process/analyze and control display of information/data such as to support the entire pre-surgery and post-surgery processes of obtaining of the biometrical properties of the hand(s) of each surgeon as well as the determination of the positioning of the surgical instrument being used at each given timeframe or moment during the surgical procedure. Therefore, the processor 110 may include one or more of the following modules, components or units:
a pre-surgery module 111 which may be operatively and electronically associated with a surgeon database (DB) 116A, an instruments DB 116B and optionally also with a patients DB 116C as well as with the sensing unit 120. For each surgeon, the pre-surgery module 111 may be configured to receive sensor data from the sensing unit 120 for determining one or more biometrical properties of the hand of the surgeon ad optionally also for measuring and/or receiving surgical instruments'properties such as instruments indication codes/indicators, dimensions and/or topography of each surgical instrument of a specific type and size, etc. and optionally generate one or more 3D hand models of the surgeon and store the measured and generated surgeon related information/data in the surgeons DB 116A. The pre-surgery module 111 may also be configured to determine and store (in the surgeons DB 116A) relative properties of the surgeon such as relative positioning of a edge of each surgical instrument in respect to one or more parts/points of the hand of the specific surgeon, wherein the determination of the relative properties is done based on pre-surgery obtained imaged/models of the surgeon's hand with and/or without the surgical instruments and optionally also based on known and retrievable properties of the surgical instruments;
a surgery module 112 configured to assist the specific surgeon during the surgical procedure for a body part 51 of a patient 50, that may be based on the personal biometrical properties pf the hand(s) of the specific surgeon and based on ongoing (real time or periodic) measuring of the surgeon's hand 10 during the surgery itself. The surgery module 112 may also be configured to generate ongoing/periodic display of a combined image illustrating the positioning of the surgical instrument 30 used by the surgeon or part of the instrument 30 such as its edge 33 and the tissue of the body part 51 that is being handled by the surgical instrument over the 3D coordinate system such that the surgeon can navigate, operate or change the instrument's position/functionality mode, etc. based on the combined image and relative positioning of the surgical instrument; and
(optionally) a training module 113 configured to train and/or test each surgeon for each specific required surgical procedure for improving and/or assessing skills level of the specific surgeon in each trained/tested surgical procedure e.g., by using real, artificial, phantom or virtual body part and/or surgical instrument(s) displayed by the system 100;

An interface and display module 118 for controlled display of a combined image showing the estimated position of the surgical instrument in respect to the tissue being operated within the 3D coordinate system, controlled display/output of messages to the surgery team in real time including, for example visual and/or auditory messages such as alerting and/or guiding messages/instructions etc.

According to some embodiments, the processor 110 and/or the remote server 140 may include a “surgery plans DB” 114 which may include for each surgery type (associated with a specific medical purpose and a specific body part and tissue(s)), one or more surgery stages each stage associated with a specific surgical instrument and one or more operation manners/modes thereof. This stored information may be used by the surgery and/or pre-surgery modules 111/112 for determining which instrument is currently used by the surgeon and how to generate based also thereon, the combined image of the body part and surgical instrument for real time surgeon's instrument-navigation assistance.

According to some embodiments, the processor 110 may also be configured to track the current stage of the specific surgical procedure, in real time or near real time, and to determine whether this stage is performed in a satisfying manner, e.g., by comparing detectable properties such as parameters values or features of detected actions of the surgeon and/or instrument used thereby and/or detected/estimated actual effect/results over the body part tissue, responsivity of the operated tissue/body part etc., with preset known equivalent detectable properties that may be retrievable from another DB such as a surgery plans DB 114, which may include surgical procedure's plans and expected stages and performances'related detectable properties.

According to some embodiments, the processor 110 may also be configured to further assist the surgeon and the surgery team, during the surgical procedure, by outputting guiding information such as guiding instructions optionally based on user input (e.g., requiring retrieval and display of steps/instructions information of a particular stage of the specific surgical procedure and/or based detected/determined surgery-stage and performances properties of the actual surgery. The display of the instructions or guidelines information may be visual and/or auditory.

According to some embodiments, the system 100 may also be configured to output alerts if and when detected performances of the surgeon, responsivity of the patient and/or detected performances of the instrument being used are not found to be satisfying. The alerts may be outputted through the display device 130, through auditory output device(s), and/or through display(s) of one or more other external end devices. According to some embodiments, the system 100 may also be configured for controlling the surgical instrument held by the surgeon and/or other devices used through the surgical procedure, for example, also enabling controllably disable the functioning/operation of the surgical instrument, upon detection of an alarming situation, detected by analyzing the hand movements of the surgeon and/or of the positioning/location of the tip of the surgical instrument in respect to the tissue of the body part that is to be operated, etc.

According to some embodiments, some of the modules described in reference to FIG. 2 as performable and operable via a local processor 110 may be additionally or alternatively performable and operable via one or more remote or external computation and/or processing devices or systems such as via one or more remote servers such as remote server 140.

According to some embodiments, the same sensing unit 120 may be used to perform measurements of the biometrical properties of surgeons'hands as well as to perform ongoing measuring of the surgeon's hand(s) and optionally also of the patient and/or body part thereof being operated, during the occurrence of the surgical procedure, using the same one or more sensors of the same sensing unit 120 or different sensors of the same sensing unit 120.

Alternatively, different one or more sensors and/or at least one different sensing unit may be used separately for the obtainment of the biometrical properties of each surgeon than the sensing unit(s) being used during the surgical procedure for the ongoing measuring of the hand of the surgeon during the surgical procedure.

According to some embodiments the one or more sensors of each sensing unit such as sensing unit 120 may include, for example one or more of the following sensors:
an optical sensor such as a camera, a laser detector, a three-point laser, a spectrometry system, etc., an imaging system such as a CT device, an MRI device, an ultrasound device, etc., and the like and may be embedded in systems that enable adjustable positioning of the patient such as in a system using an Artis Zeego® device.

According to some embodiments at least one of the surgical instruments used for at least one of the surgical procedures may configured for detection of one or more physical properties of the body part such as, for example, an endoscopic device including a camera and/or an illuminator for illuminating the operated body part or part thereof, an ultrasound transducer device, and the like.

According to some embodiments, the analysis of any measured/obtained data including, for example the analysis of the sensor data used for obtainment of the surgeon's biometrical properties and/or sensor data analysis for ongoing determination of the relative position of the surgical instrument, may be carried out by using one or more designated computer algorithms or programs that may optionally be designed as machine-learning and/or artificial intelligence (AI) algorithms, where the system 100 may be also designed for acquired and determined data accumulation and storage, such that the machine-learning/AI algorithms are designed to learn hand movements and/other surgery related behavioral features of each surgeon and optionally responsivity of different body parts or tissues thereof to different instruments and/or instrument-utilizations, and to adjust surgery plans and/or sensing/measuring techniques and/or sensor data processing/analysis characteristics and/or programs, according to the learned surgery related behavioral features.

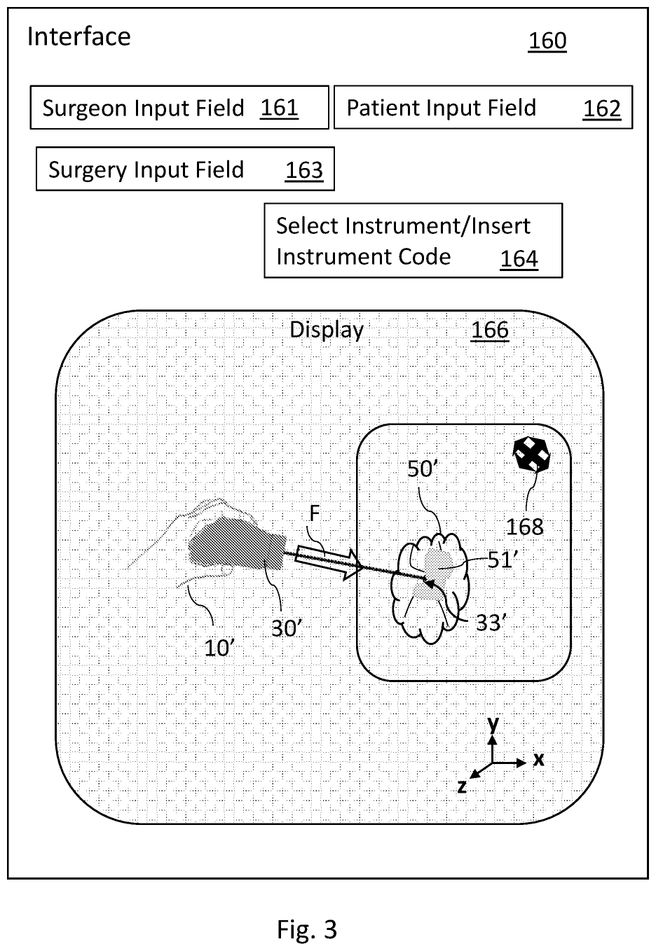

Reference is now made to FIG. 3 schematically illustrating one optional interface display 160 including:
a surgeon input filed 161 enabling a user to input a surgeon input data that will enable identification of the specific surgeon that will be carrying out a specific surgical procedure for easy retrieval, based on this input data, the one or more models and/or other biometrical properties data of the specific identified surgeon;
a patient input filed 162 enabling a user to input a patient input data that will enable identification or retrieval of relevant patient's details such as name, age, height, BMI, gender medical conditions, medical background and history etc. as well as information that is directly associated with the specific surgery such as images (e.g., ultrasound, X-ray, Zeego®, CT and/or MRI images) of the body part (tissue/organ) to be operated and the like;
a surgery input filed 163 enabling a user to input information/data that enables identification of the specific surgical procedure that the patient is to be having by the specific identified surgeon.

The three input fields 161-163 provided by the system 100 and data/information provided to the system 100 therethrough enable retrieval of one or more of: surgeon's hand biometrical properties; body part estimated configuration and positioning in the 3D coordinate system, surgical instrument(s) information for instruments that will be used during the specific identified surgical procedure.

According to some embodiments, as shown in FIG. 3, the interface 160 may also include one or more display zones such as imagery display zone 166 for example, for visually displaying of an updated combined image or model of the surgical instrument 30′ being used or an edge 33′ thereof and the body part 50′ being operated or a part 51′ thereof in a perspective that shows their relative position and optionally also additional indicators such as a direction indicator F showing the updated point vector directionality of the tip 33′ of the instrument 30′, an indication of the predefined 3D coordinate system xyz vectors, etc. According to some embodiments, the display 166 may also include one or more virtual control tools such as control tool 168 enabling adjusting of the display of the combined image such as enabling rotating the combined image, zooming in and out of a specific selectable display area, showing or marking the areas of interest, etc.

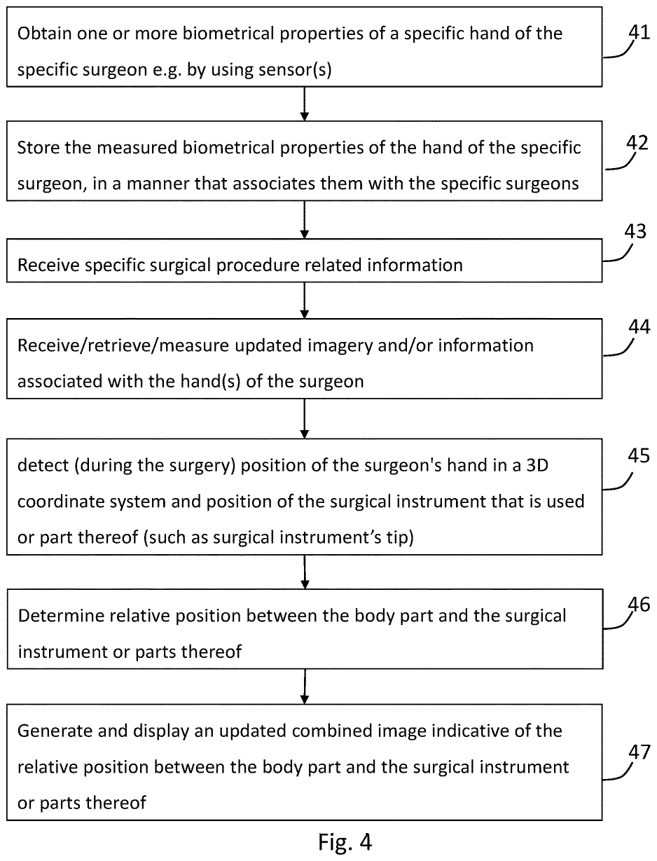

Reference is now made to FIG. 4 showing a flowchart, which schematically illustrate a method or a process for assisting surgeons in surgical navigation of surgical instruments through an inner body part, the method comprising at least:
obtaining one or more biometrical properties of a hand of a specific surgeon, measured by using one or more sensors 41;
storing the obtained (e.g., measured and/or estimated) biometrical properties in one or more storage mediums 42; and
at each given moment or timeframe of a surgical procedure conducted by the specific surgeon:
receiving and/or retrieving information associated with the specific surgical procedure to be performed 43 including for example:
   at least one updated image of a body part to be operated, at a specific perspective of the body part within a predefined coordinate system;
   information associated with the patient' and/or the body part to be operated;
   information associated with surgical instruments required for the surgical procedure such as functionality, dimensions, etc.;
   surgery plan;
retrieving biometric properties of the hand(s) of the specific surgeon 44 such as 3D models of one of the surgeon's hands (that is used for holding the surgical instruments during the surgery), in various postures and/or models of the hand when holding specific surgical instruments required for the specific surgical procedure, fingers thicknesses in various states, etc.;
detecting (e.g., in real time) one or more updated properties of the hand of the specific surgeon during the surgical procedure 45 and determine an updated hand position of at least part of the hand of the specific surgeon within a 3D coordinate system, based on both measured updated properties of the surgeon's hand and on the retrieved biometrical properties of the hand of the specific surgeon, where the updated hand position may be used to determine positioning and/or directionality of an edge (tip) of the surgical instrument currently used by the specific surgeon in the 3D coordinate system;
determining updated relative position between the surgeon's hand and/or surgical instrument and the body part or parts thereof 46, e.g., based on received updated image of the body part, the determined updated hand position of the specific surgeon and determined position of the surgical instrument, within the 3D coordinate system; and
generating and displaying of an updated combined image (such as a 3D mode or 2D image) indicative of the determined updated relative position of the surgical instrument or part thereof and the body part or part thereof 47.

The above method/process enables using at least one hand of the specific surgeon as a personalized dynamic marker for assisting the surgeon in navigation of each surgical instrument held by that hand of the specific surgeon, during the surgical procedure of the body part.

The above-described method steps 41-47 may be performed by using the system 100 described above or by any other system that enables any type of sensing of properties that an be used to obtain the biometrical properties of hands of each surgeon as well as ongoing sensing/detection of the surgery and particularly sensing/detection of the surgeon's hand during the surgical procedure.

It is to be understood that the above disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

### EXAMPLES

Example 1 is a method for assisting surgeons in surgical navigation of surgical instruments through an inner body part, the method may include, for example, at least:
(i) obtaining one or more biometrical properties of a hand of a specific surgeon, measured by using one or more sensors; and
at each given moment or time frame of a surgical procedure conducted by the specific surgeon:
(ii) receiving at least one updated image of a body part to be operated, at a specific perspective of the body part within a predefined coordinate system;
   measuring one or more updated properties of the hand of the specific surgeon during the surgical procedure and determine an updated hand position of at least part of the hand of the specific surgeon within the coordinate system, based on the measured updated properties and the obtained biometrical properties of the hand of the specific surgeon; and determining updated relative position between the surgeon's hand and the body part, based on received updated image of the body part and determined updated hand position of the specific surgeon within the coordinate system, thereby using the hand of the specific surgeon as a personalized dynamic marker for assisting the surgeon in navigation of at least one surgical instrument held by the hand of the specific surgeon, during the surgical procedure of the body part.

In example 2, the subject matter of example 1 may include, wherein the biometrical properties of the hand of the specific surgeon comprise one or more of:
topography of the hand of the specific surgeon;
one or more topographies of typical hand postures of the hand of the specific surgeon when holding one or more types and sizes of surgical instruments;
typical movements of the hand of the specific surgeon when holding and using one or more types and sizes of surgical instruments;
distances between different parts of the hand of the specific surgeon;
relative position between one or more parts of the hand of the specific surgeon and one or more parts of one or more surgical instruments;
width, length and/or thickness of one or more parts of the surgeon's hand;
relations between the posture of the hand of the specific surgeon and a relative directionality of the surgical instrument held by the hand of the specific surgeon or part thereof;
changes in one or more of the one or more biometrical properties of the hand of the specific surgeon when under various postures, positions and/or conditions.

In example 3, the subject matter of any one or more of examples 1 to 2 may include, wherein the method further comprises generating a 3D model of at least part of the hand of the specific surgeon based on the obtained one or more biometrical hand properties, wherein the determination of the updated relative position between the hand of the surgeon and the body part is used for determining corresponding updated position of at least part of a surgical instrument held by the specific surgeon in respect to at least an area of the body part.

In example 4, the subject matter of example 3 may include, wherein the method further comprises generating and displaying of an updated combined image showing a relative position of the surgical instrument being used, or a part thereof, in respect to the body part within the predefined coordinate system, according to the determined updated relative position between the hand of the surgeon and the body part.

In example 5, the subject matter of any one or more of examples 1 to 4 may include, wherein the surgical procedure and the body part being operated is virtual, simulated or real.

In example 6, the subject matter of any one or more of examples 1 to 5 may include, wherein the detection of the updated relative position between the body part and the at least one part of the surgeon's hand is done in real time or near real time.

In example 7, the subject matter of any one or more of examples 1 to 6 may include, wherein the method further comprises assessing progress and/or quality of the surgical procedure being carried out by the specific surgeon, based on real time and/or ongoing assessment of the relative position of a surgical instrument held by the specific surgeon in respect to the body part, wherein the assessment of the relative positioning of the surgical instrument is done based on the determined relative position of the hand of the specific surgeon.

In example 8, the subject matter of any one or more of examples 1 to 7 may include, wherein the method further comprises ongoing displaying of a 3D image of the hand of the specific surgeon and the body part in their determined relative position therebetween.

In example 9, the subject matter of any one or more of examples 1 to 8 may include, wherein the biometric properties of the hand of the specific surgeon are measured by using one or more optical sensors comprising one or more: cameras, 3D optical sensor, 3D points cloud sensor, laser scanners, 2D optical sensors.

In example 10, the subject matter of example 9 may include, wherein the updated image of the body part comprises a 3D model of the respective body part constructed by using one or more previously measured and/or scanned images of the respective body part.

In example 11, the subject matter of any one or more of examples 1 to 10 may include, wherein the method further comprises visually displaying an updated image indicative of the determined updated relative position of the hand of the specific surgeon, a surgical instrument held and used by the hand of the specific surgeon and/or a part of the surgical instrument held and used by the hand of the specific surgeon, in respect to the received updated position of the body part.

In example 12, the subject matter of example 11 may include, wherein the updated image is a 3D updated image.

In example 13, the subject matter of any one or more of examples 1 to 12 may include, wherein the received updated image of the body part is obtained by using at least one imaging system.

In example 14, the subject matter of example 13 may include, wherein the at least one imaging system comprises one or more of: a computer tomography (CT) system, systems that use one or more patient's positioning adjustment devices, a magnetic resonance imaging (MRI) system, an isotopic tomography system, an ultrasound system, a scanning system, a X-ray system, a nano-rods based optical detection system, a camera based system, an endoscopy based system.

In example 15, the subject matter of any one or more of examples 13 to 14 may include, wherein the imaging system for obtaining the updated image of the body part is used in an ongoing manner during the surgical procedure for providing updated real time or near real time updated images of the body part and its updated position within a predefined three-dimensional or two-dimensional coordinate system.

In example 16, the subject matter of any one or more of examples 1 to 15 may include, wherein the hand of the specific surgeon operating the surgical instrument is fully or partially located externally to the body part.

In example 17, the subject matter of any one or more of examples 1 to 16 may include, wherein the method further comprises receiving user input via a designated user interface, the user input being indicative at least of one or more of:
surgical instrument information indicative or enabling retrieval of one or more properties of the surgical instrument to be used during the surgical procedure or part thereof;
body part information indicative of updated or previously acquired one or more images of the body part to be operated during the surgical procedure or part thereof;
surgeon information indicative of the specific surgeon performing the surgical procedure and/or the specific one or more of his/her hands that will be used during the surgical procedure.

In example 18, the subject matter of any one or more of examples 1 to 17 may include, wherein the method further comprises detection of changes in the biometrical properties of the hand of the specific surgeon the changes being one or more of:
pressure level applied by the specific surgeon onto the surgical instrument or part thereof during the surgical procedure;
position and/or posture of the hand of the specific surgeon;
wherein the one or more detected changes are used for determining one or more of:
directionality of the surgical instrument being used or part thereof and/or the direction of the overall force applied by the surgical instrument;
orientation of the surgical instrument or part thereof within a predefined 3D coordinate system;
current and/or predicted operational behavior characteristics of the surgical instrument;
operational present and/or predicted future status, stage and/or state;
errors in the surgical procedures;
   current and/or predicted surgery behavior of the specific surgeon.

Example 19 is a system for assisting surgeons in surgical navigation of surgical instruments through an inner body part, the system comprising at least:
(i) one or more sensors at least for obtaining of one or more biometrical properties of a hand of a specific surgeon;
(ii) at least one processor configured at least for:
receiving an updated image of a body part at a specific perspective of the body part within a predefined coordinate system;
detecting one or more updated properties of the hand of the specific surgeon during the surgical procedure and determine an updated hand position of at least part of the hand of the specific surgeon, based on the detected updated properties and the obtained biometrical properties of the hand of the specific surgeon; and
determining updated relative position between the surgeon's hand and the body part, based on received updated image of the body and determined updated hand position of the specific surgeon, thereby using the hand of the specific surgeon as a personalized dynamic marker for assisting in guiding the specific surgeon through surgical procedure of the body part.

In example 20, the subject matter of example 19 may include, wherein the at least one processor is further configured to generate and display, via one or more display devices, an updated combined image showing a relative position of the surgical instrument being used, or a part thereof, in respect to the body part within the predefined coordinate system, according to the determined updated relative position between the hand of the surgeon and the body part.

In example 21, the subject matter of any one or more of examples 19 to 20 may include, wherein the biometrical properties of the hand of the specific surgeon comprise one or more of:
topography of the hand of the specific surgeon that is typically used by the specific surgeon to hold surgical instruments;
one or more topographies of typical hand postures of the hand of the specific surgeon when holding one or more types and sizes of surgical instruments;
typical movements of the hand of the specific surgeon when holding and using one or more types and sizes of surgical instruments;
typical distances between different parts of the hand of the specific surgeon;
typical relative position between one or more parts of the hand of the specific surgeon and one or more parts of one or more surgical instruments;
typical relations between the posture of the hand of the specific surgeon and a relative directionality of the surgical instrument held by the hand of the specific surgeon or part thereof and/or direction of an overall force applied by the surgical instrument;
typical changes in one or more of the one or more biometrical properties of the hand of the specific surgeon when under various postures, positions and/or conditions.

In example 22, the subject matter of any one or more of examples 19 to 21 may include, wherein the at least one processor is further configured for generating a 3D model of at least part of the hand of the specific surgeon based on the obtained one or more biometrical hand properties, wherein the determination of the updated relative position between the hand of the surgeon and the body part is used for determining corresponding updated position of at least part of a surgical instrument held by the specific surgeon in respect to at least an area of the body part.

In example 23, the subject matter of any one or more of examples 19 to 22 may include, wherein the surgical procedure and the body part being operated is virtual, simulated or real.

In example 24, the subject matter of any one or more of examples 19 to 23 may include, wherein the detection of the updated relative position between the body part and the at least one part of the surgeon's hand is done in real time or near real time.

In example 25, the subject matter of any one or more of examples 19 to 24 may include, wherein the at least one processor is further configured for assessing progress and/or quality of the surgical procedure being carried out by the specific surgeon, based on real time and/or ongoing assessment of the relative position of a surgical instrument held by the specific surgeon in respect to the body part, wherein the assessment of the relative positioning of the surgical instrument is done based on the determined relative position of the hand of the specific surgeon.

In example 26, the subject matter of any one or more of examples 19 to 25 may include, wherein the system is further configured for ongoing displaying of a 3D image of the hand of the specific surgeon and the body part in their determined relative position therebetween.

In example 27, the subject matter of any one or more of examples 19 to 26 may include, wherein the biometric properties of the hand of the specific surgeon are obtained by using one or more optical sensors comprising one or more: camera, 3D optical sensor, 3D point cloud sensor, laser scanner, 2D optical sensors, ultrasound device, MRI system, CT system.

In example 28, the subject matter of example 27 may include, wherein the updated image the body part comprises a 3D model of the respective body part constructed by using one or more previously measured and/or scanned images of the respective body part.

In example 29, the subject matter of any one or more of examples 19 to 28 may include, wherein the processor is further configured to generate and visually display an updated image indicative of the determined updated relative position of the hand of the specific surgeon, a surgical instrument held and used by the hand of the specific surgeon and/or a part of the surgical instrument held and used by the hand of the specific surgeon, in respect to the received updated position of the body part.

In example 30, the subject matter of example 29 may include, wherein the updated image is a 3D updated image.

In example 31, the subject matter of any one or more of examples 19 to 30 may include, wherein the received updated image of the body part is obtained by using at least one imaging system.

In example 32, the subject matter of example 31 may include, wherein the at least one imaging system comprises one or more of: a computer tomography (CT) system, a magnetic resonance imaging (MRI) system, an isotopic tomography system, an ultrasound system, a scanning system, a X-ray system, a nano-rods based optical detection system, a camera based system, an endoscopy based system.

In example 33, the subject matter of any one or more of examples 31 to 32 may include, wherein the imaging system for obtaining the updated image of the body part is used in an ongoing manner during the surgical procedure for providing updated real time or near real time updated images of the body part and its updated position within a predefined three-dimensional or two-dimensional coordinate system.

In example 34, the subject matter of any one or more of examples 19 to 33 may include, wherein the hand of the specific surgeon operating the surgical instrument is fully or partially located externally to the body part during surgery.

In example 35, the subject matter of any one or more of examples 19 to 34 may include, wherein the system further comprises a designated user interface for receiving user input and displaying of information therethrough, the user input being indicative at least of one or more of:
surgical instrument information indicative or enabling retrieval of one or more properties of the surgical instrument to be used during the surgical procedure or part thereof;
body part information indicative of updated or previously acquired one or more images of the body part to be operated during the surgical procedure or part thereof;
surgeon information indicative of the specific surgeon performing the surgical procedure and/or the specific one or more of his/her hands that will be used during the surgical procedure.

In example 36, the subject matter of any one or more of examples 19 to 35 may include, wherein the at least one processor is further configured to detect changes in the biometrical properties of the hand of the specific surgeon the changes being one or more of:
pressure level applied by the specific surgeon onto the surgical instrument or part thereof during the surgical procedure;
position and/or posture of the hand of the specific surgeon;
wherein the one or more detected changes are used for determining one or more of:
directionality of the surgical instrument being used or directionality of part thereof;
orientation of the surgical instrument or part thereof within a predefined 3D coordinate system;
measured and/or predicted current operational behavior characteristics of the surgical instrument;
operational present and/or predicted future status, stage and/or state;
errors in the surgical procedures;
measured and/or predicted surgery behavior of the specific surgeon.

In example 37, the subject matter of any one or more of examples 19 to 36 may include, wherein the at least one processor is further configured to operate one or more machine-learning and/or artificial intelligence (AI) algorithms for learning surgical procedures related behavioral features over time and adjusting analysis and/or processing characteristics, based on learned behavioral features.

1. A method for assisting surgeons in surgical navigation of surgical instruments through an inner body part, the method comprising at least:
(i) obtaining one or more biometrical properties of a hand of a specific surgeon, measured by using one or more sensors; and
at each given moment or time frame of a surgical procedure conducted by the specific surgeon:
(ii) receiving at least one updated image of a body part to be operated, at a specific perspective of the body part within a predefined coordinate system;
(ii) measuring one or more updated properties of the hand of the specific surgeon during the surgical procedure and determine an updated hand position of at least part of the hand of the specific surgeon within the coordinate system, based on the measured updated properties and the obtained biometrical properties of the hand of the specific surgeon; and
(iii) determining updated relative position between the surgeon's hand and the body part, based on received updated image of the body part and determined updated hand position of the specific surgeon within the coordinate system, thereby using the hand of the specific surgeon as a personalized dynamic marker for assisting the surgeon in navigation of at least one surgical instrument held by the hand of the specific surgeon, during the surgical procedure of the body part.

2. (canceled)

3. The method of claim 1 further comprising generating a 3D model of at least part of the hand of the specific surgeon based on the obtained one or more biometrical hand properties, wherein the determination of the updated relative position between the hand of the surgeon and the body part is used for determining corresponding updated position of at least part of a surgical instrument held by the specific surgeon in respect to at least an area of the body part.

4. The method of claim 3 further comprising generating and displaying of an updated combined image showing a relative position of the surgical instrument being used, or a part thereof, in respect to the body part within the predefined coordinate system, according to the determined updated relative position between the hand of the surgeon and the body part.

5. (canceled)

6. (canceled)

7. The method of claim 1 further comprising assessing progress and/or quality of the surgical procedure being carried out by the specific surgeon, based on real time and/or ongoing assessment of the relative position of a surgical instrument held by the specific surgeon in respect to the body part, wherein the assessment of the relative positioning of the surgical instrument is done based on the determined relative position of the hand of the specific surgeon.

8. The method of claim 1 further comprising ongoing displaying of a 3D image of the hand of the specific surgeon and the body part in their determined relative position therebetween.

9. (canceled)

10. (canceled)

11. The method of claim 1 further comprising visually displaying an updated image indicative of the determined updated relative position of the hand of the specific surgeon, a surgical instrument held and used by the hand of the specific surgeon and/or a part of the surgical instrument held and used by the hand of the specific surgeon, in respect to the received updated position of the body part.

12. (canceled)

13. (canceled)

14. (canceled)

15. (canceled)

16. The method of claim 1, wherein the hand of the specific surgeon operating the surgical instrument is fully or partially located externally to the body part.

17. The method of claim 1 further comprising receiving user input via a designated user interface, the user input being indicative at least of one or more of:
surgical instrument information indicative or enabling retrieval of one or more properties of the surgical instrument to be used during the surgical procedure or part thereof;
body part information indicative of updated or previously acquired one or more images of the body part to be operated during the surgical procedure or part thereof;
surgeon information indicative of the specific surgeon performing the surgical procedure and/or the specific one or more of his/her hands that will be used during the surgical procedure.

18. The method of claim 1 further comprising detection of changes in the biometrical properties of the hand of the specific surgeon the changes being one or more of:
pressure level applied by the specific surgeon onto the surgical instrument or part thereof during the surgical procedure;
position and/or posture of the hand of the specific surgeon;
wherein the one or more detected changes are used for determining one or more of:
directionality of the surgical instrument being used or part thereof and/or the direction of the overall force applied by the surgical instrument;
orientation of the surgical instrument or part thereof within a predefined 3D coordinate system;
current and/or predicted operational behavior characteristics of the surgical instrument;
operational present and/or predicted future status, stage and/or state;
errors in the surgical procedures;
current and/or predicted surgery behavior of the specific surgeon.

19. A system for assisting surgeons in surgical navigation of surgical instruments through an inner body part, the system comprising at least:
(i) one or more sensors at least for obtaining of one or more biometrical properties of a hand of a specific surgeon;
(ii) at least one processor configured at least for:
receiving an updated image of a body part at a specific perspective of the body part within a predefined coordinate system;
detecting one or more updated properties of the hand of the specific surgeon during the surgical procedure and determine an updated hand position of at least part of the hand of the specific surgeon, based on the detected updated properties and the obtained biometrical properties of the hand of the specific surgeon; and
determining updated relative position between the surgeon's hand and the body part, based on received updated image of the body and determined updated hand position of the specific surgeon, thereby using the hand of the specific surgeon as a personalized dynamic marker for assisting in guiding the specific surgeon through surgical procedure of the body part.

20. The system of claim 19, wherein the at least one processor is further configured to generate and display, via one or more display devices, an updated combined image showing a relative position of the surgical instrument being used, or a part thereof, in respect to the body part within the predefined coordinate system, according to the determined updated relative position between the hand of the surgeon and the body part.

21. (canceled)

22. The system of claim 19, wherein the at least one processor is further configured for generating a 3D model of at least part of the hand of the specific surgeon based on the obtained one or more biometrical hand properties, wherein the determination of the updated relative position between the hand of the surgeon and the body part is used for determining corresponding updated position of at least part of a surgical instrument held by the specific surgeon in respect to at least an area of the body part.

23. (canceled)

24. (canceled)

25. The system of claim 19, wherein the at least one processor is further configured for assessing progress and/or quality of the surgical procedure being carried out by the specific surgeon, based on real time and/or ongoing assessment of the relative position of a surgical instrument held by the specific surgeon in respect to the body part, wherein the assessment of the relative positioning of the surgical instrument is done based on the determined relative position of the hand of the specific surgeon.

26. The system of claim 19 is further configured for ongoing displaying of a 3D image of the hand of the specific surgeon and the body part in their determined relative position therebetween.

27. (canceled)

28. (canceled)

29. The system of claim 19, wherein the processor is further configured to generate and visually display an updated image indicative of the determined updated relative position of the hand of the specific surgeon, a surgical instrument held and used by the hand of the specific surgeon and/or a part of the surgical instrument held and used by the hand of the specific surgeon, in respect to the received updated position of the body part.

30. (canceled)

31. (canceled)

32. (canceled)

33. (canceled)

34. The system of claim 19, wherein the hand of the specific surgeon operating the surgical instrument is fully or partially located externally to the body part during surgery.

35. The system of claim 19 further comprises a designated user interface for receiving user input and displaying of information therethrough, the user input being indicative at least of one or more of:
surgical instrument information indicative or enabling retrieval of one or more properties of the surgical instrument to be used during the surgical procedure or part thereof;
body part information indicative of updated or previously acquired one or more images of the body part to be operated during the surgical procedure or part thereof;
surgeon information indicative of the specific surgeon performing the surgical procedure and/or the specific one or more of his/her hands that will be used during the surgical procedure;

36. The system of claim 19, wherein the at least one processor is further configured to detect changes in the biometrical properties of the hand of the specific surgeon the changes being one or more of:
pressure level applied by the specific surgeon onto the surgical instrument or part thereof during the surgical procedure;
position and/or posture of the hand of the specific surgeon;
wherein the one or more detected changes are used for determining one or more of:
directionality of the surgical instrument being used or directionality of part thereof;
orientation of the surgical instrument or part thereof within a predefined 3D coordinate system;
measured and/or predicted current operational behavior characteristics of the surgical instrument;
operational present and/or predicted future status, stage and/or state;
errors in the surgical procedures;
measured and/or predicted surgery behavior of the specific surgeon.

37. The system of claim 19, wherein the at least one processor is further configured to operate one or more machine-learning and/or artificial intelligence (AI) algorithms for learning surgical procedures related behavioral features over time and adjusting analysis and/or processing characteristics, based on learned.

38. A non-transitory computer readable storage medium having computer readable program code embodied therewith, the computer readable program code, executable by at least one processing circuitry of a computer to perform a method for assisting surgeons in surgical navigation of surgical instruments through an inner body part, the method comprising at least:
(i) obtaining one or more biometrical properties of a hand of a specific surgeon, measured by using one or more sensors; and
at each given moment or time frame of a surgical procedure conducted by the specific surgeon:
(ii) receiving at least one updated image of a body part to be operated, at a specific perspective of the body part within a predefined coordinate system;
(ii) measuring one or more updated properties of the hand of the specific surgeon during the surgical procedure and determine an updated hand position of at least part of the hand of the specific surgeon within the coordinate system, based on the measured updated properties and the obtained biometrical properties of the hand of the specific surgeon; and
(iii) determining updated relative position between the surgeon's hand and the body part, based on received updated image of the body part and determined updated hand position of the specific surgeon within the coordinate system, thereby using the hand of the specific surgeon as a personalized dynamic marker for assisting the surgeon in navigation of at least one surgical instrument held by the hand of the specific surgeon, during the surgical procedure of the body part.
